(19) United States
(12) Patent Application Publication
(10) Pub. No.: US 2020/0129424 A1
(43) Pub. Date: Apr. 30, 2020

(54) MICRONEEDLE SYSTEM FOR APPLYING GLUCAGON-LIKE PEPTIDE ANALOGUES

(71) Applicant: LTS LOHMANN THERAPIE-SYSTEME AG, Andernach (DE)

(72) Inventors: Andreas HENNING, Koblenz (DE); Heiko SPILGIES, Koblenz (DE)

(21) Appl. No.: 16/619,611

(22) PCT Filed: Jun. 6, 2018
(86) PCT No.: PCT/EP2018/064919

§ 371 Date: Dec. 5, 2019

(30) Foreign Application Priority Data
Jun. 7, 2017 (DE) 10 2017 112 573.6

(51) Int. Cl. A61K 9/00; A61M 37/00; A61K 38/26; A61K 47/32; A61K 47/10; A61K 47/36; A61K 47/26

(52) U.S. Cl. CPC A61K 9/0021; A61M 37/0015; A61K 38/26; A61M 2037/0023; A61K 47/10; A61K 47/36; A61K 47/26; A61K 47/32

(57) ABSTRACT

The present invention relates to a microneedle system (MNS) for intradermal application in a controlled release of glucagon-like peptide analogues.

The present invention relates to a microneedle system (MNS) for intradermal application in a controlled release of glucagon-like peptide analogues.

The glucagon-like peptide GLP-1 (glucagon like peptide-1) is an intestinal hormone and is formed with GLP-2 in intestinal cells. GLP-1 is an insulinotropic peptide and regulates the blood sugar level. Its physiological task lies in maintaining a normal glucose homeostasis and an even energy balance. GLP-1 inhibits glucagon secretion and delays gastric emptying. GLP-1 is not suitable as a therapeutic agent, since it has an extremely short half-life period of approximately one minute.

If the structure of GLP-1 is slightly changed, however, the degrading peptidase DDP IV remains ineffective. Even marginal molecular changes, such as the replacement of an amino acid, make the molecule resistant to the peptidase. The GLP-1 analogues developed until now act, in part, more strongly than the endogenous GLP-1 and are used as antidiabetics, but must be applied subcutaneously.

Such antidiabetics based on glucagon-like peptide analogues or antagonists—also referred to as incretin mimetics—are in particular exenatide, liraglutide and lixisenatide (hereinafter: glucagon-like peptide analogue) and bind to the GLP-1 receptor. Such glucagon-like peptide analogues have up to 50 amino acids and a molar mass up to 5 kDa. The half-life period is usually more than 12 hours.

Exenatide (C₁₈₄H₂₈₂N₅₀O₆₀S, Mr=4186.6 Da) is a synthetic peptide which consists of 39 amino acids.

Liraglutide or γ-L-glutamoyl(N-a-hexadecanoyl)-Lys26, Arg34-GLP-1(7-37) is a branched-chain peptide (C₁₇₂H₂₆₅N₄₃O₅₁, Mr=3751.2 Da) which consists of 39 amino acids.

Lixisenatide (C₂₁₅H₃₄₇N₆₁O₆₅S, Mr=4858.49 Da) is a synthetic peptide which consists of 44 amino acids.

Exenatide is preferred in accordance with the invention.

The skin consists of a number of layers. The outermost skin layer, the Stratum Corneum, has known barrier properties in order to prevent an infiltration of foreign substances into the body and an escape of endogenous substances from the body.

The Stratum Corneum, which is a complex structure of compacted keratotic cell residues with a thickness of approximately 10-30 micrometres, to this end forms a water-tight membrane for protection of the body. The natural impermeability of the Stratum Corneum prevents the administration of most pharmaceutical agents and other substances through the skin within the scope of an intradermal application. It is particularly difficult to introduce proteins through the skin.

Microneedle systems (MNS), which consist of a microneedle array (MNA) and optionally further components, may press the microneedles (also referred to as the main penetration elements) of the array (MNA) against the application point on the skin by means of a compressive force so as to penetrate the Stratum Corneum and in this way produce a fluid channel, such that an active substance may be applied intradermally. Such microneedle arrays (MNA) in microneedle systems (MNS) and production thereof are described in the prior art (WO2004000389A1).

It is also known that MNS may be used for controlled release. In particular for glucagon-like peptide analogues there is a high need for controlled release, in particular for more than 12 hours (h) or even 24 h, such that an antidiabetic effect may be achieved or assisted for a day.

U.S. Pat. No. 9,320,878 B2 describes a controlled release of exenatide (claim 4) by means of an MNS based on polyvinyl alcohols (PVA). U.S. Pat. No. 9,320,878 B2 asserts that a controlled release based on polyvinylpyrrolidone (PVP) is not possible.

PVP, however, is preferred over PVA for influencing the release of active substances and for this purpose has a greater suitability.

The object of the present invention is therefore to enable an intradermal application in a controlled release of glucagon-like peptide analogues by means of an MNS containing an MNA based on a suitable formulation.

This object is achieved in accordance with the invention by a microneedle system (MNS) containing an MNA according to claim 1, wherein the glucagon-like peptide analogue together with the PVP is the subject of the formulation of the MNA and therefore is inherent to the MNA or forms a unit with the MNA. To this end the MNA may be formed integrally or in one part.

The subject of the invention is therefore a microneedle system containing an MNA for use in the intradermal application for controlled release comprising or consisting of a formulation of PVP and at least one glucagon-like peptide analogue.

The invention therefore comprises a product with a microneedle array comprising or consisting of a formulation of PVP and at least one glucagon-like peptide analogue for use in the intradermal application for controlled release.

Such a product is, for example, a medicinal product or antidiabetic comprising a protruding microneedle array for controlled release of at least one glucagon-like peptide analogue in an intradermal application. Said medicinal product is used for the prophylaxis and treatment of diabetes, type II diabetes, insulin resistance, high blood pressure, obesity (Adipositas, Obesitas), and metabolic syndrome.

The term “intradermal application” (referred to synonymously as “intracutaneous application”) in accordance with the invention describes the administration of substances (here: glucagon-like peptide analogues) from the MNA into the skin and requires the skin to be pierced by the microneedles.

The term “controlled release” within the scope of this invention means that the active substance (here: glucagon-like peptide analogue) is released or dispensed continuously over a time period of more than 2 h, preferably more than 12 h, in particular 24 h. The active substances are not released suddenly or immediately (immediate release).

The invention therefore relates to a product for use in the intradermal application for controlled release, wherein the time period of the controlled release is more than 2 h, preferably more than 12 h, in particular 24 h.

A dose of up to 50 μg/MNA of glucagon-like peptide analogue may particularly advantageously be applied to humans.

This allows the advantageous one-time application of a daily dose of glucagon-like peptide analogue.

A further subject of the invention is a method for the controlled release of at least one glucagon-like peptide analogue in an intradermal application having a microneedle array comprising or consisting of a formulation of PVP and at least one glucagon-like peptide analogue.

A further subject of the invention is a method for performing an intradermal application for the controlled release of at least one glucagon-like peptide analogue comprising
a) fixing of a microneedle system according to the invention to the skin,
b) penetration of a microneedle array comprising a formulation of PVP and at least one glucagon-like peptide analogue into the skin.

In a further embodiment the formulation may consist of any PVP with a relative molecular mass of more than 10,000 Da. The proportion of PVP (% by weight) in the formulation may be 50-99%, in particular 70-90%, preferably 75-80%. In particular the PVP content is dependent on the residual water. Typical residual water contents may be 5-20%, in particular 12-18%. A residual water content of 16% results in a PVP content of approximately 76% or possibly lower.

Furthermore, the formulation may contain auxiliaries and additives, such as
a.) binders or emulsifiers, for example carboxymethyl cellulose, alginates, gelatines,
b.) humectants, for example glycerol, urea, trehalose,
c.) wetting agents, for example cetyl alcohol, glycerol monostearate,
d.) antioxidants, such as ascorbic acid, vitamin E.

Preferred, however, are poloxamers, alginates and polysorbate (Tween).

The proportion of poloxamer in the formulation may be 2-7%. The proportion of alginate in the formulation may be 2-7%. The proportion of polysorbates in the formulation may be 0.1-1%. microneedle system containing a microneedle array is configured with an applicator. Such applicators advantageously allow the activation of a pressure mechanism for penetrating the microneedle array into the skin or the Stratum Corneum (for example see WO2003091602A2, WO2016162449A1).

In a further embodiment the applicator system containing a microneedle array may be configured with conventional functional items which allow a fixing on the skin as well as simple handling for exerting pressure onto the akin and in particular may contain at least one fixing means.

Within the scope of this invention an applicator system is a system which contains a device which causes the microneedle array to be provided on the skin and applied intradermally for administration of a glucagon-like peptide analogue.

The applicator system in an advantageous preferred embodiment may comprise a trigger device which is controlled electrically or mechanically. The applicator system for example may have a plunger which positions or applies the microneedle array on/to the skin, such that the microneedles penetrate into the skin.

The trigger device may comprise, for example, a pump, a syringe or a spring, such that a plunger may impact with sufficient energy. The piston may be of any form and nature and should primarily ensure that the microneedle array is provided from a first position into a second position on the skin for administration of the active substances.

The applicator system may also comprises a push button or thread.

In accordance with a further embodiment the microneedle array may contain fixing means which are preferably fastened to the skin of a patient or test subject with the aid of an adhesive glue strip or patch (also referred to as a needle patch). Highly viscous substances which stick to the skin when pressed on briefly and lightly (what are known as pressure-sensitive adhesives or PSAs) are suitable as adhesives. They have high cohesion and adhesion forces. For example, adhesives based on poly(meth)acrylates, based on polyisobutylenes or based on silicones may be used. In a further embodiment the fixing means may consist of a band, elastic band, rubber or belt. A secure fixing to the body may be achieved with the aid of such fixing means.

Such applicator systems allow the secure positioning of the MNA according to the invention for the controlled release of glucagon-like peptide analogues.

The following examples and figures are intended to explain the invention in greater detail, without limiting the invention.

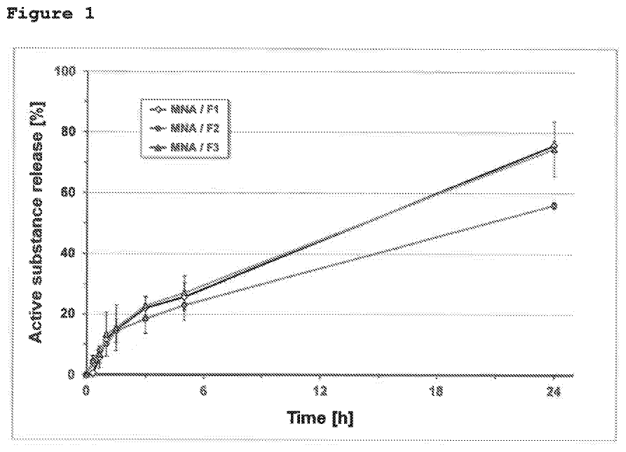

FIG. 1 shows in-vitro results for formulations F1, F2 and F3, which demonstrate a slowed, time-controlled release of the active substance exenatide over a time period of up to 24 hours. The formulations are identified in Table 1.

TABLE 1
Composition of formulations F1, F2 and F3

| | | Active substance load |
|---|---|---|
| | Ingredients/pharm. | (Exenatide/microneedle |
| Formulation | auxiliaries | array) |
| F1 | PVP (K29/32)*; AGT2; | 50 μg/MNA |
| | Poloxamer | |
| F2 | PVP (K90)**; AGT; | 50 μg/MNA |
| | ascorbic acid | |
| F3 | PVP (K29/32); AGT | 50 μg/MNA |

*PVP (K29/32) = medium molecular weight PVP (~58 kDa)
**PVP(K90) = high molecular weight PVP (~1.3 MDa)
2AGT = sodium alginate, glycerol, Tween (80)

### EXAMPLE 1

The composition of the microneedles includes the following ingredients/pharm. auxiliaries:
polyvinylpyrrolidone (PVP) type 1: MW 20-60 kDa; type 2: >1000 kDa
poloxamer (Pluronic F-68)
sodium alginate
glycerol
polyoxyethylene sorbitan monooleate (polysorbate, Tween-80)

After drying, some of the water remains in the array so as to form the matrix structure together with the polymer (PVP).

A suitable embodiment of the formulation for the active substance exenatide with 50 μg is as follows:

TABLE 2
Composition of a formulation

| | Composition - | Composition - |
|---|---|---|
| Ingredient | liquid solution - | after drying* - |
| PVP | 20% | 73.9% |
| Poloxamer | 1% | 3.7% |
| Sodium alginate | 1% | 3.7% |
| Glycerol (85% sol.) | 1% | 3.1% (calculated as |
| | | anhydrous glycerol) |
| Tween-80 (10% sol.) | 1% | 0.4% (calculated as |
| | | anhydrous Polysorbate 80) |
| Water | 75.8% | 15% |
| Exenatide | 0.2% | 0.2% |

*practical drying to 12-16% residual moisture

1.-12. (canceled)

13. A microneedle array comprising a formulation of 50-99% by weight of polyvinylpyrrolidone and at least one glucagon-like peptide analogue for use in the intradermal application for controlled release.

14. A product having a microneedle array comprising a formulation of 50-99% by weight of polyvinylpyrrolidone and at least one glucagon-like peptide analogue for use in the intradermal application for controlled release.

15. The product according to claim 14 for use in the intradermal application for controlled release for the prophylaxis and treatment of diabetes, type II diabetes, insulin resistance, high blood pressure, Adipositas, and metabolic syndrome.

16. The microneedle array for use in the intradermal application for controlled release according to claim 13, characterised in that the glucagon-like peptide analogues are selected from the group consisting of exenatide, liraglutide, lixisenatide, and combinations thereof.

17. The microneedle array for use in the intradermal application for controlled release according to claim 13, characterised in that the time period of the controlled release is more than 2 h.

18. The microneedle array for use in the intradermal application for controlled release according to claim 13, characterised in that a formulation contains 50 to 99% by weight of polyvinylpyrrolidone and further auxiliaries and additives.

19. The microneedle array for use in the intradermal application for controlled release according to claim 18, wherein the formulation comprises 2-7% poloxamer or 2-7% alginates or 0.1-1% polysorbates.

20. The microneedle array for use in the intradermal application for controlled release according to claim 13, wherein the formulation comprises 70-90% by weight of polyvinylpyrrolidone.

21. The microneedle array for use in the intradermal application for controlled release according to claim 13, characterised in that a dose of up to 50 μg/MNA glucagon-like peptide analogue is applied.

22. The product for use in the intradermal application for controlled release according to claim 14, comprising an applicator system.

23. A method for carrying out an intradermal application for the controlled release of at least one glucagon-like peptide analogue in an intradermal application having a microneedle array comprising a formulation of 50-99% polyvinylpyrrolidone and at least one glucagon-like peptide analogue.

24. The method for carrying out an intradermal application for the controlled release of at least one glucagon-like peptide analogue according to claim 23 comprising
a.) fixing of a microneedle system according to the invention to the skin,
b.) penetration of a microneedle array comprising a formulation of PVP and at least one glucagon-like peptide analogue into the skin.

25. The microneedle array for use in the intradermal application for controlled release according to claim 13, characterised in that the time period of the controlled release is more than more than 12 h.

26. The microneedle array for use in the intradermal application for controlled release according to claim 13, wherein the formulation comprises 75-80% by weight of polyvinylpyrrolidone.
